(19) United States
(12) Patent Application Publication
(10) Pub. No.: US 2003/0008335 A1
(43) Pub. Date: Jan. 9, 2003

(54) Biosensor for drug candidates

(76) Inventors: Kenneth A. Marx, Francestown, NH (US); Susan J. Braunhut, Wellesley, MA (US); Tiean Zhou, Lowell, MA (US); Anne Montrone, Raymond, NH (US)

Correspondence Address: J. PETER FASSE, Fish & Richardson P.C., 225 Franklin Street, Boston, MA, 02110-2804, US

(21) Appl. No.: 10/144,193

(22) Filed: May 13, 2002

Related U.S. Application Data
(60) Provisional application No. 60/290,306, filed on May 11, 2001.

(51) Int. Cl. G01N 33/574; C12M 1/34

(52) U.S. Cl. 435/7.23; 435/287.1

(57) ABSTRACT

The Quartz Crystal Microbalance (QCM) creates a piezoelectric biosensor utilizing living endothelial cells (ECs) as the biological signal transduction element. ECs adhere to the hydrophilically treated gold QCM surface under growth media containing serum. The EC QCM biosensor can be used for the study of EC attachment and to detect EC cytoskeletal alterations. The cellular biosensor can be used for real time identification or screening of classes of biologically active drugs or biological macromolecules that affect cellular attachment, regardless of their molecular mechanism of action.

### CROSS-REFERENCE TO RELATED APLICATION

This application claims the benefit of Provisional Patent Application Serial No. 60/290,306, filed on May 11, 2001, which is incorporated herein by reference in its entirety.

### TECHNICAL FIELD

This invention relates to biosensors for drug candidates.

### BACKGROUND

The Quartz Crystal Microbalance (QCM) was developed and applied initially to the measurement of mass binding to the quartz surface from chemical species in a gas phase. The possibility of solution based QCM has been a more recent development, and it is largely used as a tool in analytical electrochemistry. Using the Sauerbray equation [1], the QCM is capable of sensitively measuring mass changes associated with liquid-solid interfacial phenomena, particularly at electrodes [2-5]. Surface bound elastic mass can be distinguished from viscoelastic behavior of bound mass or solution viscosity-density effects on the crystal frequency, f, and resistance, R, values using established techniques [6-8].

Biosensors have been created using the QCM piezoelectric signal transduction mechanism, in which a range of biological macromolecules have been incorporated into the sensing system design [9-22]. Infrequent reports have appeared investigating whole cells studied at the QCM surface. Surface adherent cell types previously studied have included: osteoblasts, human platelets, MDCK I and II cells, 3T3 cells, CERO cells, CHO and MKE epithelial cells and microbial biofilms [23-30]. These studies establish the basic principle that adherent cells produce a reversible QCM frequency shift. However, the considerable variability in reported delta-f shift values in most of the studies are not explained. Overall, cells adhering to the QCM surface, do not act as elastic masses obeying the Sauerbray Equation [1].

Endothelial cells (ECs) represent an important cell type in the body, forming a continuous monolayer of cells that line the blood vessels [31]. In large vessels closest to the heart, the EC monolayer resides upon a basement membrane shared by multiple layers of smooth muscle cells [32]. In microvessels, the number of extramural cell layers is reduced. However, these vessels are also lined by a continuous monolayer of ECs. ECs are involved in the regulation of vessel diameter, blood flow and the movement of gases, nutrients and metabolic waste between the plasma and interstitial spaces. ECs are tightly growth regulated, rarely proliferating in vivo. In some tissues, ECs have been estimated to divide only once every three years [34].

ECs of microvessels are dynamic and can be rapidly stimulated by angiogenic signals to become mobilized and to proliferate during wound healing or in association with particular pathologies [35]. The endothelium is required to alter its cell shape and its binding to the underlying extracellular matrix (ECM) during this change from a non-growing to a growing, migratory state. Coupling of these cells with the matrix is via cytoskeletal elements linked to the plasma membrane at inner cytoplasmic domains termed focal adhesion complexes (FACs) and is mediated by integrin receptors that span the plasma membrane [36]. Integrin receptors and FACs possess extracellular domains that can bind directly to sequences found within the individual ECM molecules. Mass re-distribution or changes in the cytoskeleton occur when ECs change growth state or respond to chemicals that alter cytoskeletal properties. Little is known about these changes and how whole cell properties of ECs are altered.

Microtubules are known to be affected significantly in their dynamic properties and their steady state structure within cells as a consequence of the binding of a class of molecules that largely interact with the major microtubule structural protein subunit called tubulin. The drug nocodazole, in the nM to μM concentration range, has been demonstrated to bind tubulin in vivo and to depress the dynamic instability properties of microtubules and eventually to disassemble these structures in a variety of eukaryotic cells [38] including ECs.

### SUMMARY

The invention is based on the discovery that certain cells can be adhered to a quartz crystal microbalance (QCM) and used to analyze the efficacy of drug candidates, such as anticancer drug candidates.

According to one aspect of the invention, a biosensor detects biological molecules in a quartz crystal microbalance (QCM), where the biosensor includes an endothelical cell (EC) matrix used as biological signal transduction elements and a piezoelectric mechanism for signal transduction to access attached EC cells as biological elements in the QCM biosensor.

One or more of the following features may also be included.

In certain embodiments, the EC adheres to a hydrophilically treated gold QCM surface under growth media containing serum. Additionally, the biosensor detects EC cytoskeletal alterations.

As another feature, the biosensor can be used to screen classes of biologically active drugs or biological macromolecules affecting cellular attachment, regardless of their molecular mechanism of action.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The invention provides several advantages. This novel cellular biosensor monitors different adherent cells in cell biology research projects where the viscoelastic properties of the cellular cytoskeleton and attachment may be altered by a particular experimental manipulation. Other benefits include the simple screening of the effects of small molecule drugs and other therapeutics on adherent cells.

Additionally, the new biosensor can be used to analyze agents targeted for signal transduction elements such as the cytoskeleton, membrane bound integrins, and the extracellular matrix. Moreover, the new cellular biosensor can be adapted to the requirements of high throughput screening found in the pharmaceutical industry, as array formats of the QCM are developed for use.

Other features and advantages of the invention will be apparent from the following detailed description, and from the claims.

### DESCRIPTION OF DRAWINGS

FIGS. 1A and B are schematics of a Cell QCM Biosensor and signal transduction elements and an entire Cell QCM Biosensor Measurement System.

FIG. 2 is a graph illustrating the EC QCM biosensor recorded Δf shift values (absolute values) at 20 hours after addition of cells. These Δf shift values are plotted against the number of ECs determined to be firmly attached to the QCM surface, via trypsinization and electronic counting, at the conclusion of each Δf shift experiment. The data has been fit to a hyperbolic curve with R²=0.82.

FIG. 3 is a graph illustrating the EC QCM biosensor recorded ΔR shift values at 20 hours after addition of cells. These AR shift values are plotted against the number of ECs determined to be firmly attached to the QCM surface, via trypsinization and electronic counting, at the conclusion of each ΔR shift experiment. The data has been fit to a hyperbolic curve with R²=0.83.

FIG. 4 is a graph illustrating the time-dependent behavior of the EC QCM biosensor created by the addition of 20,000 ECs at first arrowhead. The Δf and ΔR values were recorded continuously until steady state properties were observed. At second arrowhead, nocodazole was added to a final 2.0 μM concentration.

FIG. 5 is a dose curve of Δf vs. log nocodazole concentration where Δf maximum values have been plotted. A three component sigmoid curve has been fit to the data where R²=0.91.

FIG. 6 is a dose curve of ΔR vs. log nocodazole concentration where ΔR maximum values have been plotted. A three component sigmoid curve has been fit to the data where R²=0.96.

FIGS. 7A to 7F are a series of fluorescence light microscopy images of ECs grown to their steady state at 24 hours on LabTeks, treated with nocodazole and then stained for actin:

FIG. 7A, control; FIG. 7B, 0.33 μM; FIG. 7C, 1 μM; FIG. 7D, 2 μM; FIG. 7E, 6 μM; and FIG. 7F, 15 μM.

FIGS. 8A and 8B are fluorescence light microscopy images of ECs grown to their steady state at 24 hours on QCM surfaces treated with nocodazole and then stained for actin: FIG. 8A, control and FIG. 8B, 15 μM.

### DETAILED DESCRIPTION

Little has been done to use the sensitive quantitative capabilities of the QCM to correlate measured f and R values with bound cell number and type, or to investigate the behavior of adherent cells in response to chemical, biological or physical changes in their environment. This is ironic because cells represent biologically evolved systems that possess all the attributes of smart materials. In some sense they represent the ultimate smart material. That is, they possess the abilities of self-assembly, self-multiplication, self-repair, self-degradation, redundancy, self-diagnosis, learning and prediction/notification. These are embodied in the evolved molecular systems of cells. They are expressed in various ways, such as cells being responsive to their environment in real time, homeostatic in response to certain properties or stimuli, capable of integration of multiple complex functions or states such as molecular recognition, discrimination, feedback, standby. In terms of our ability to harness this evolved smart behavior in a biosensor format, what is necessary is a molecular mechanism to transduce information, such as a specific analyte concentration, that cells sense in their environment. We demonstrate here that the QCM can provide a piezoelectric mechanism for signal transduction that allows us access to, the attached ECs as the biological element in this EC QCM biosensor.

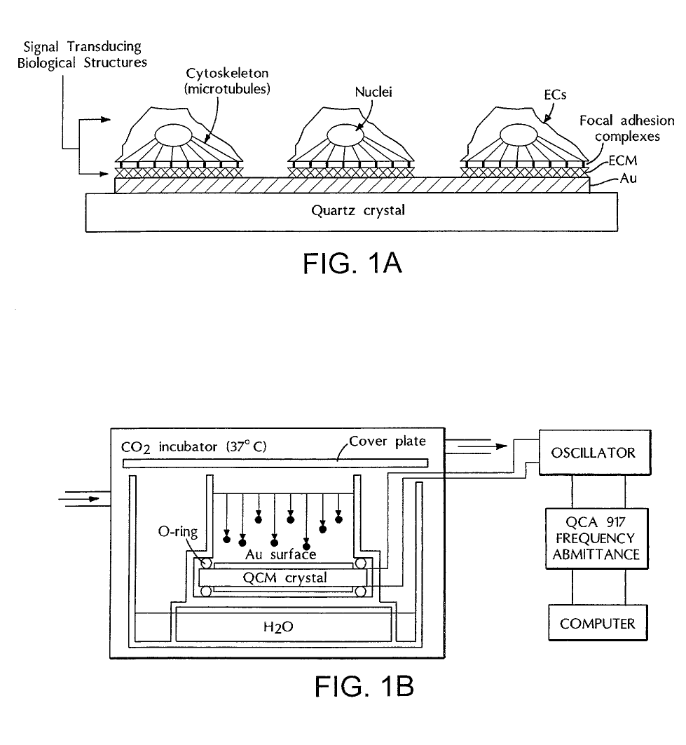

In FIG. 1A, we schematically depict the signal transduction region of this biosensor, in which the ECs stably adhere to the QCM at the steady state condition. At the steady state the ECs have synthesized ECM upon the gold surface, to which they subsequently attach themselves via FACs. Internally, the cytoskeleton is coupled to the FACs. A principle structural component of the cytoskeleton is an array of microtubules. The cytoskeleton allows cells to stretch and spread, acquiring a greater area on the attachment surface. We identify the putative biological signal transduction elements here to be the network ranging from the microtubules to the ECM contacting the gold surface.

We have described, in a preliminary study, the time dependent sequence of initial contact, immediate adhesion and spreading of normal bovine aortic ECs as they sediment to, contact and spread on the gold QCM surface [39]. A number of technical issues needed to be overcome for the QCM to function reproducibly as a transducing device for this biosensor. We have reported on a number of parameters [40] affecting normal bovine endothelial cells, including: varying O-ring toxicity for ECs and an observed long range frequency oscillation artifact, due to water evaporation, which was eliminated via modifications of the experimental set-up. Of great importance, we demonstrated the necessity of relating the QCM Δf and ΔR crystal shifts to the final cell number bound, as determined by electronic counting of the cell number requiring trypsinization to be removed from the gold QCM surface at the conclusion of the experiment, rather than relating Δf and ΔR to the number of cells added to the QCM cell. We also demonstrated that growth stimulation of ECs by FGF on the QCM surface resulted in a Δf shift that reflects initial cell shape changes and later the increase in cell number bound to the QCM surface. Lastly, we demonstrated that from the first few minutes ECs bind to the gold QCM surface through their establishment of a stable attached steady state at 24 hour, a dramatic increase occurs in the energy dissipation properties of the QCM solution-surface interface, which goes from an initial pure liquid like viscoelastic behavior to a greater energy dissipation state.

In the present application, we build upon the prior investigations, which use bovine aortic ECs, a normal, limited lifespan cell type derived from freshly excised bovine aortas. Here, we have switched cells to utilize commercially available tissue culture bovine ECs. These ECs, incorporated into the EC QCM biosensor, exhibit Δf and ΔR shift calibration curves that demonstrate saturation effects as a function of the cell numbers requiring trypsinization for removal from the gold QCM biosensor surface. At a particular fixed number of ECs on the biosensor surface, we then investigated the behavior of the EC QCM biosensor as a function of adding nocodazole to the growth media of the cells. We observed dose dependent Δf and ΔR shift effects with nocodazole concentration, in the range from 0.11-15 μM. This correlated with fluorescence light microscope observations of changes in EC intercellular contacts and progressive cell rounding, beginning around 330 nM and saturating by 6 μM.

### Quartz Crystal Microbalance for Cell Adhesion

The details of how these measurements were made have been presented previously [40]. Basically, an AT cut quartz crystal of resonant frequency 8.85 MHz with gold electrode (5 mm diameter)was used in a cylindrical Teflon cell (Seiko EG&G). The crystal was sandwiched between two silicone O-rings to allow only one side of the electrode to be exposed to the media and serum containing solution (FIG. 1B). The QCM device was placed within a large petri dish, filled with distilled water, at a level well above the water surface. This water reservoir allowed humidity to be maintained, preventing evaporation from the QCM cell. The QCM was covered with a petri dish cover plate following placement inside a 37° C. temperature regulated cell incubator. A Model QCA 917 Quartz Crystal Analyzer System (Seiko EG&G), comprised of a Main Unit and Oscillator, was used for the simultaneous measurement of the resonant frequency (f) and resonance admittance (A).

Before assembly in the well holder, the gold QCM surface was treated chemically to render it hydrophilic [41]. A drop of 1:3 H₂O₂ (30%): H₂SO₄ at 80° C. was placed upon and covered the gold surface for 5 minutes, followed by rinsing with distilled water and drying under N₂. This procedure was repeated three times. After sterilization and washing with water and PBS, 52 μl of media and 10% Calf Serum (CS) was added onto the QCM electrode and the entire holder was put into a humidified CO₂ incubator controlled at 10% CO₂. The f and admittance (A) values were automatically monitored at 1 minute intervals using a PC and WinWedge 32, version 3.0 Software (TAL Technologies®) and the stable values at 2 hour were taken as reference values before the addition of cells. After 2 hours, 250 μl media +10% CS containing a specific number of cells was added evenly to the medium surface. Then f and A values were automatically monitored during the process of EC sedimentation to the crystal surface, and during their impact and spreading over a 20 hr period to achieve steady state attachment. In the data reduction phase, values of A, in μS were converted into motional resistance, R, in units of Ω, using the relationship R=10⁶/A. Nocodazole achieved its final concentration by being added in a pre-warned 50 μl volume of media and serum, following removal of 50 μl from the top of the EC QCM biosensor well holder. Following the conclusion of each experiment, trypsinable ECs adhering to the gold QCM surface were determined as described below.

### Cell Culture, Cell Attachment to the QCM and Removal via Trypsinization

Bovine aortic endothelial cells (BAEs) were obtained from Clonetics, Inc., and were maintained as stock cultures in DMEM-10% CS, as previously described [40]. BAEs were not used beyond passage 22. For experimental treatments, ECs were trypsinized, washed with PBS, and precise counts of the number of cells were made using an electronic cell counter made by Coulter, Inc., following resuspension in full media (250 μl). At the end of a QCM experiment, cells firmly attached were determined via trypsinization assay and an electronic counter. This protocol involved the following steps. Media was collected from the QCM (M). The QCM was gently washed with 100 μl PBS (W). After 125 μl trypsin (0.05% w/v trypsin, 0.53 mM EDTA, in Hank's Buffered salt solution) was added and incubated for 6 min. at 37° C., this fraction was collected (T₁). Following a second trypsinization wash, identical to the first, this fraction was collected (T₂). Aliquots from all of the four steps (M, W, T₁, T₂) were electronically counted using Coulter counting. After trypsinization, the QCM electrode was washed successively with PBS, water, ethanol, then detached from the well holder. It was then cleaned sequentially with a number of reagents to remove all residual ECM and cell debris, as we have previously described [40]. Cleaned in this way, the QCM was used repeatedly with good reproducibility of its initial f and R values, due to complete protein removal from the QCM surface.

### QCM Simulation Using ECs in Multichamber LabTek Slides Treated with Nocodazole

To acquire detailed information about the change in cell shape and attachment while ECs were exposed to varying nocodazole concentrations, we performed a simulation experiment. 76,400 ECs were plated into multichamber LabTek slides and were allowed to attach for 24 hrs in normal growth media. This number of cells simulates the cell surface density in the QCM device. 50 μL of media was then removed and replaced with 50 μL of media with or without concentrated nocodazole. In duplicate, cells either received no nocodazole (control) or final concentrations of 0.33, 1, 2, 6 or 15 μM nocodazole. After four hours, the cells were fixed and stained to reveal the actin microfilament arrangement within the cells, as described below. These studies were performed to detect the shape and attachment changes in ECs that would roughly correspond to a time when the QCM detected maximal shifts in f and R values. We chose to localize actin within the cells because at low doses of nocodazole we found that the microtubules began to depolymerize leading to a diffuse staining pattern in the cells when cells were stained using anti-tubulin antibodies specific to microtubules (obtained from Sigma Chem Co.). Staining actin revealed an intact element of the cytoskeleton allowing us to visualize the cell shape and degree of spreading.

### Actin Localization and Immunofluorescent Microscopy

For actin localization in the multichamber wells or in the QCM device, media was aspirated and replaced with 3.5% formaldehyde. The cells were fixed for 15 mins at room temperature, washed with PBS and then incubated with rhodamine-labeled phalloidin (0.165 μM in PBS) for 30 mins at room temperature. The cells were then washed with PBS and coverslipped with Cytoseal mounting medium (VWR Scientific). The cells were examined using an Olympus BH2 CTD microscope, and the images were digitized using a CCD camera and imported into Photoshop 5.0 of Adobe® and using a Gateways® P11-390 computer.

### Cellular Origin and Magnitude of Δf and ΔR Shifts

When ECs are added to the QCM, they sediment through the media and serum contained within the cylindrical QCM cell holder to the gold surface on the upper face of the QCM crystal. As the ECs contact the surface and adhere, they cause an initial steep decrease in the f values and an increase in the R values of the QCM crystal over the first hr. As the ECs form a stable attachment with time, the Δf and ΔR shift magnitudes peak over the first 2-3 hrs, then slowly decrease. They reach steady state values by 15 hours, following addition, that reflect the numbers of firmly attached ECs and their elaborated cellular attachment system responsible for their fully spread state on the crystal surface. This EC state is what we presented schematically in FIG. 1A. We have described this entire process of EC attachment to the QCM surface previously, using a primary culture EC, the bovine aortic EC [39-42].

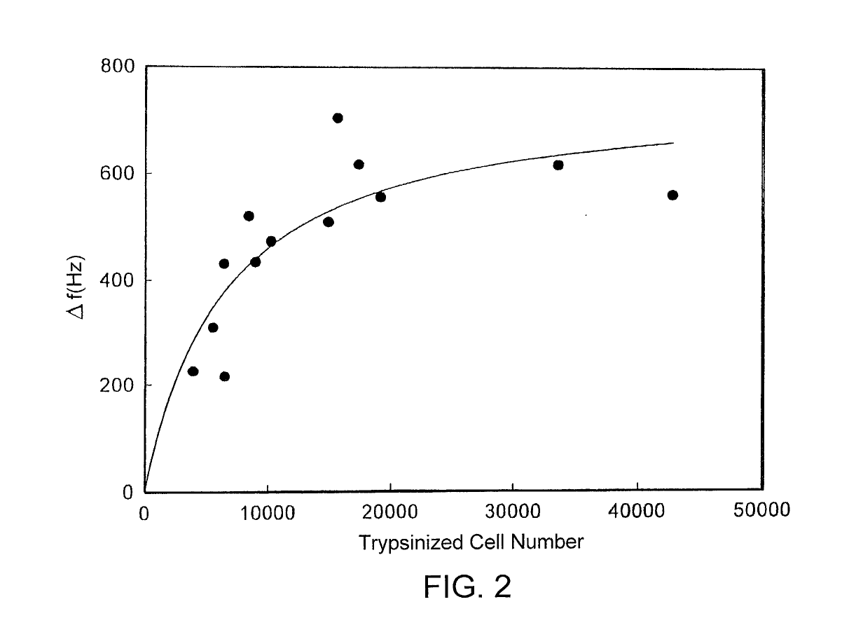

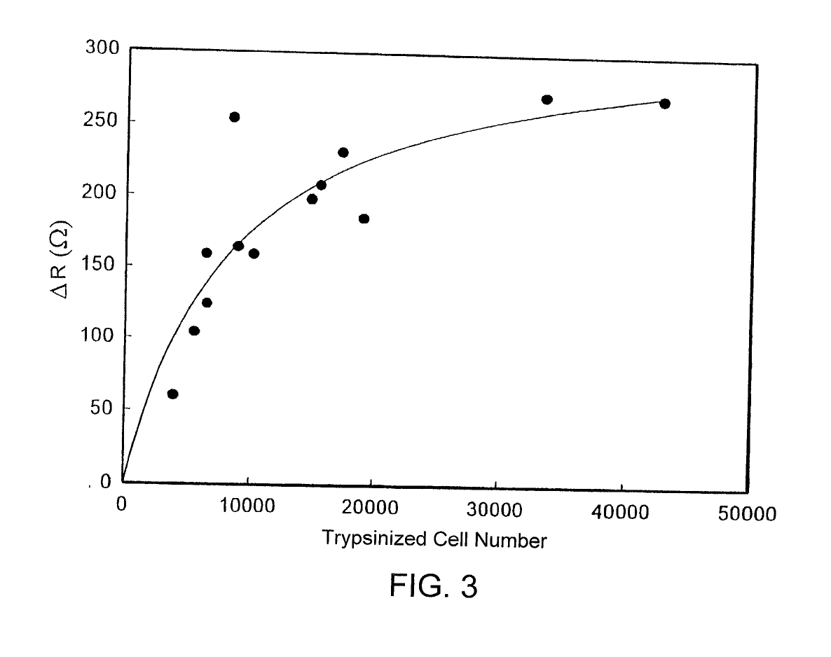

In FIGS. 2 and 3, we illustrate how the 24 hours steady state Δf and ΔR shift values vary as a function of the number of trypsinized ECs removed from the gold QCM surface at the conclusion of the experiment, which were then electronically counted. As we previously demonstrated, the Δf and ΔR shift values are determined specifically by the number of cells requiring trypsin to be removed from the surface in two trypsin incubation washes, following initial steps involving media removal and then a PBS wash [40]. In all of these studies, the removal of ECs via trypsinization resulted in the f and R values returning precisely to their original values before ECs were added. The EC calibration curves of Δf and ΔR vs. adhering cell number demonstrate an initial rise that eventually exhibits saturation behavior at higher bound EC numbers. This behavior mimics that of a non-linear binding isotherm, such as the classic Langmuir adsorption model [43]. In FIGS. 2 and 3, we carried out fits of both datasets to a hyperbolic function of the Langmuir Isotherm form, which resulted in reasonable goodness of fit values. For the regions preceding saturation behavior (5,000-15,000 bound ECs), we calculated average sensitivity values of −0.029 Hz/cell for Δf shifts and 0.012 Ω/cell for ΔR shifts from linear fits of the data. Within this presaturation range, the QCM acts as a sensor of adhering EC number.

Previous cell studies have tended to describe the QCM Δf and ΔR responses in terms of the number of cells added. We have demonstrated that a more accurate measure of the Δf and ΔR shift response is the number of trypsinizable ECs adhering to the QCM surface [40]. This fact can be understood in light of the well known cell biological phenomenon of plating efficiency. That is, less than 100% of the trypsinized cells added to a new surface will bind to and spread on that surface and remain viable. When we compare the added EC number vs. the trypsinizable ECs at the steady state condition for the EC QCM biosensor studied here, the plating efficiency of stably attached cells was found to be about 70% on average (data not shown), over the range of 5,000-50,000 added ECs. This variable plating efficiency is probably due to different passage numbers of the cells, since cells age even in culture. With each passage, their ability to be removed from one surface and re-establish themselves on a new surface is diminished. In FIGS. 2 and 3, Δf and ΔR response saturation at around 30,000 ECs adhering to the surface is related to the entire gold surface becoming effectively saturated with coverage by fully spread ECs.

### Detecting Effects of nM Nocodazole, a Microtubule Binding Drug

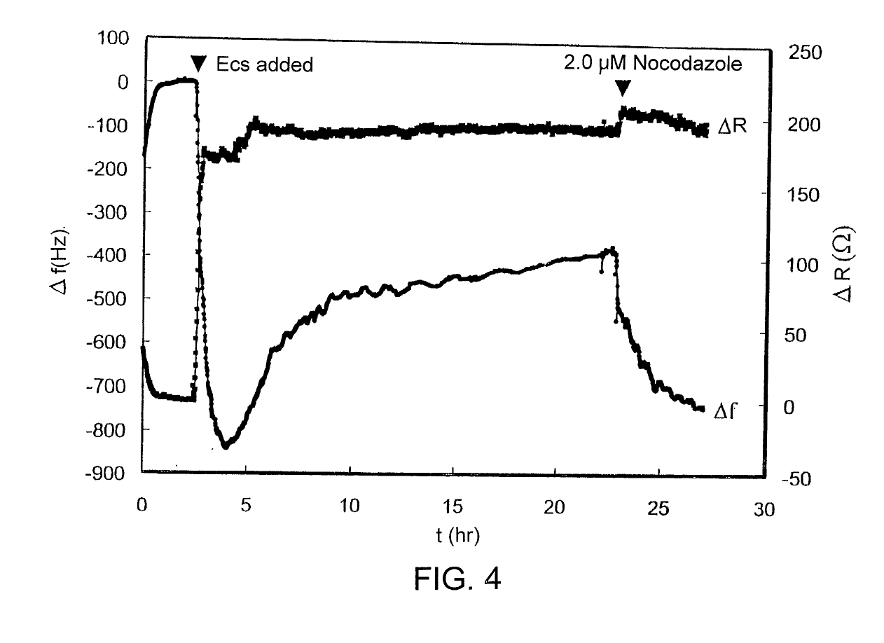

We have utilized this cellular QCM biosensor, containing 20,000 added ECs, to detect the concentration dependent effect of nocodazole, a potent microtubule binding drug, on EC morphology and cell shape. One typical example of these experiments is presented in FIG. 4. Following the initial establishment of baseline f and R values in the presence of media and serum, 20,000 ECs were added to the QCM device (at first arrowhead). This cell number was chosen because the resulting 12-16,000 attached ECs at the steady state, lies within the FIG. 2 (Δf) and FIG. 3 (ΔR) linear, pre-saturation regions. The initial decrease in f and increase in R, result from the ECs contacting and adhering to the gold surface, following their short sedimentation time through the media and serum. In independent simulation experiments, we have demonstrated that by 45 min, nearly all rounded ECs have reached the gold QCM surface [42]. The Δf and ΔR shifts reached their maxima at around 2-3 hours following EC addition. These shift maxima are due to rounded cells firmly adhering to the surface before they spread and establish the steady state adherence phenotype [39-42]. By as early as 10-15 hr following cell addition, the ECs have established steady state Δf and ΔR shifts nearly characteristic of the fully elaborated EC QCM biosensor. This reflects cells having reached a fully spread equilibrium steady state.

At the second arrowhead position, nocadazole was added to a final concentration of 2.0 μM. This dose of nocodazole has the effect of disrupting polymerized microtubules, one of the principle elements of the internal cytoskeleton of the cell. At this dose, there is a clear and significant effect of the nocodazole detected by the biosensor. In this particular experiment, the Δf drops a full 360 Hz over the next 4 hours, while a small initial maximum increase of 14 Ω is observed in ΔR followed by a slow decline. That these shifts are due specifically to the action of nocodazole was established by carrying out a control experimental volume replacement without the drug being present. Only minor changes in Δf (−20 Hz) and ΔR (2 Ω) were observed (data not shown). In contrast, after 4 hours of nocodazole treatment, the Δf and ΔR shifts resemble those of the cells at 2-3 hours post cell addition, when the ECs are known to be rounded and not yet fully spread on the surface [42]. Therefore, nocodazole treatment, which depolymerizes microtubules [38], produces an effect on the ECs causing them to restore their rounded state, as seen earlier prior to the spreading process, before microtubules in the ECs' cytoskeleton are established which links them via integrins to the ECM on the gold QCM surface.

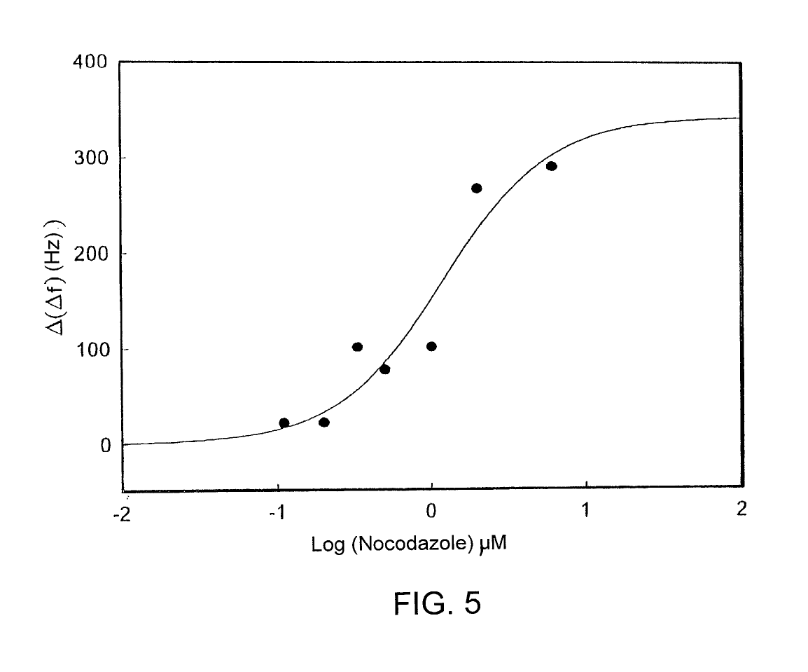

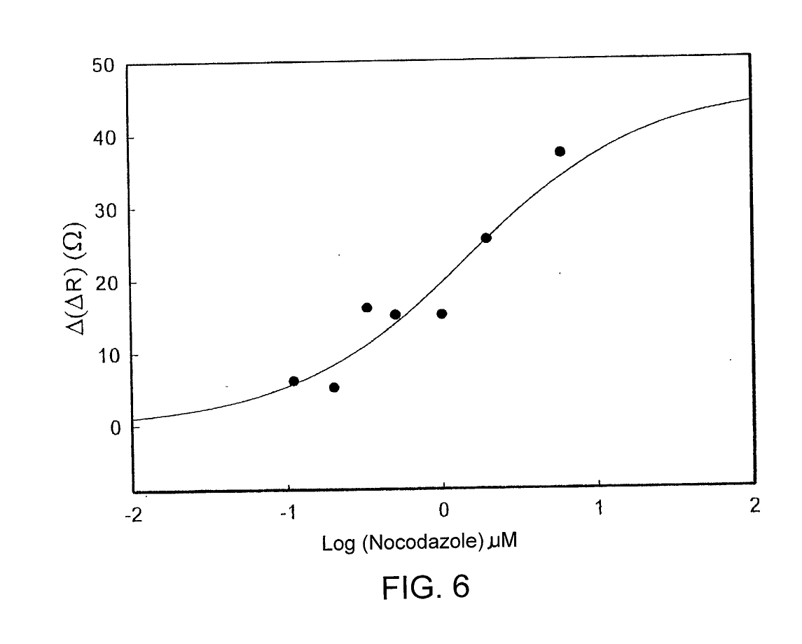

We carried out a series of separate experiments using the steady state EC QCM biosensor at a range of nocodazole concentrations between 0.11-15 μM. For each experiment, we have determined the maximum Δf decrease and ΔR increase relative to their steady state values immediately before drug addition. The Δ(Δf) and Δ(ΔR) values are plotted vs. log [Nocodazole] in FIGS. 5 and 6, respectively. These curves clearly exhibit sigmoid shapes characteristic of a nocodazole dose effect on the ECs' attachment. state. At the low end of the concentration range investigated, the magnitudes of the measured Δ(Δf) and Δ(ΔR) shifts are close to the control Δ(Δf) and Δ(ΔR)values discussed above. At the high end of the nocodazole concentration range, the shifts are clearly due to significant alterations of the attached ECs' cytoskeleton. In fact, the 15 μM datapoint was not included in this dose curve because of light microscopic evidence that ECs were beginning to detach from the crystal surface. For 15 μM, both the Δ(Δf) and Δ(ΔR) values were only 50% of that measured for the 6 μM values.

This EC QCM biosensor measures an effective change in cellular attachment in 50% of the ECs (PC₅₀) at the transition midpoint of about 900 nM for nocodazole. In particular, this PC₅₀ value and the range of our observed dose effects is within the range of measured biological effects reported for nocodazole administered to other cell types. These in view effects include interference with microtubule dynamic instability at nanomolar concentrations and then microtubule disassembly in the low micromolar concentration range [38-44].

### Fluorescence Microscopy Evidence for Cytoskeletal Rearrangement as a Function of Nocodazole Exposure

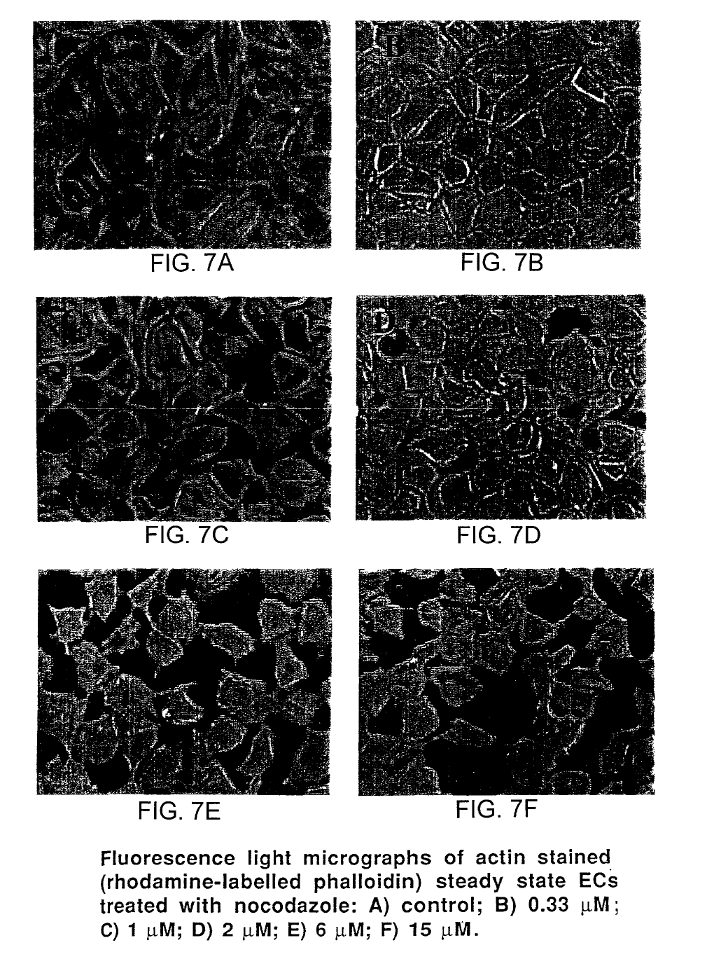

Nocodazole effects on ECs were studied in fluorescence light microscopy experiments shown in FIG. 7 simulating ECs on the QCM. These were performed in multichamber Labteks, where ECs were plated and allowed to attach for 24 hours. In parallel with nocodazole treated cells, controls then had 50 μL of media removed and then replaced with control media and 4 hours later cells were fixed and stained for actin microfilaments (panel A). These studies revealed that untreated ECs were well-spread and in a continuous monolayer with a cobblestone morphology, a characteristic growth pattern of normal ECs. The actin microfilaments were lightly stained and surrounded the nucleus and the perimeter of the cell in a normal arrangement in these control cells. In parallel, ECs were treated with varying doses of nocodazole for 4 hr and then fixed and stained. After a 0.33 μM nocodazole treatment, EC shape and morphology was altered panel). The ECs were smaller than control cells and although the cells still maintained a monolayer with close apposition of cell membranes, actin microfilaments accumulated in stress fibers at the perimeter of the cells and were brightly stained. The appearance of stress fibers is an indication that the cells are having difficulty maintaining their well spread cell shape with the loss of some microtubules. At higher doses of nocodazole, 1 and 2 μM (panels C and D respectively), the monolayer became disrupted and cells could no longer maintain their cell-cell contacts with the dissolution of the microtubule scaffold within the cells. Spaces opened between most cells and they appeared less well spread. At doses of 6 and 15 μM (panels E and F), cells occupied a similar, significantly reduced cellular area at the level of the dishes when compared with the control (panel A) and maintained few, if any, cell-cell contacts.

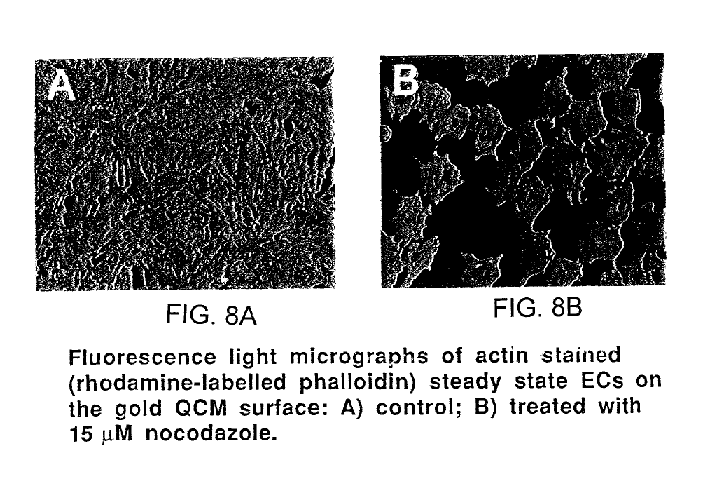

Actin localization within cells grown and or treated with nocodazole in the QCM. We wished to examine the ECs directly attached to the QCM surface with and without nocodazole treatment. Since the coils needed to be chemically fixed on the surface, we needed to sacrifice crystals for this purpose. Two different crystals were used. The first crystal was used to visualize ECs growing on the gold QCM surface at the steady state condition. At the conclusion of the QCM measurement, cells on the crystals were stained to localize actin microfilaments. In the first experiment, ECs were added to the crystal and were allowed to attach for 24 hrs to achieve their normal characteristic Δf and ΔR shifts. The cells were then fixed and stained for actin and are presented in FIG. 8, panel A. ECs grown on the crystal surface were indistinguishable from control ECs grown on conventional tissue culture plasticware (FIG. 7, panel A). ECs spread and acquired a cobblestone morphology within a monolayer when grown under the experimental conditions used for QCM.

Using the second sacrificed QCM crystal, upon which all of the nocodazole dose experiments performed in this study were carried out, ECs were added to the QCM and allowed to attach for 24 hours. The cells were then treated with nocodazole and four hours later were fixed and stained for actin. Representative cell images are presented in FIG. 8 panel B. These cells treated with nocodazole on the crystal underwent the same morphologic changes observed in the FIG. 7 QCM simulation. The cells lost cell-cell contact, occupied a smaller cellular area at the level of the crystal and acquired a rounder cell shape. These studies confirm that changes in Δf and ΔR shift values are likely to reflect alterations of cell shape and cell adhesion as a function of increased doses of nocodazole and decreased amounts of polymerized microtubules within the cells. The cellular QCM biosensor can be used successfully to detect subtle alterations of cytoskeleton and its effects on cell shape that are identical to those occurring on conventional tissue culture plasticware surfaces.

### CONCLUSION

We have created a cellular QCM biosensor that utilizes ECs in the present embodiment, as the biological signal transduction element. This EC QCM Biosensor was used to construct a dose effect curve whose midpoint value for PC₅₀=900 μM. Effects were detected by this biosensor at as low as 330 μM nocodazole. Fluorescence light microscopy images of actin stained ECs treated over the same range of nocodazole concentrations, verified the biosensor dose curve. In individual experiments at low drug concentrations, the ECs seem to be beginning to exhibit reversibility, and thus the new QCM biosensor should be reuseable.

We have previously demonstrated that ECs can attach to the gold QCM surface, to form firmly attached monolayers in cell culture medium that exhibit characteristic ΔF and ΔR shifts [40]. These shifts reveal a significant level of energy dissipative behavior by the ECs, increasing with time to reach steady state values over a 24 hour period. Changes in either the QCM effective surface bound mass of this complex system, or in the mechanical or energy dissipation behavior of these coupled signal transduction elements (cytoskeleton, FACS or ECM) can bring about corresponding changes in sensor output, expressed as alterations in the Δf and ΔR values. Therefore, any small drug molecules or large biomolecules such as proteins can be sensed by this biosensor, if they effect changes in the viscoelastic properties of ECs and the ECM via either physical effects following binding or via biochemical metabolic alterations of the cytoskeleton, integrins, or ECM. Also, we previously demonstrated that this EC based QCM biosensor is capable of long term biosensing. We studied Fibroblast Growth Factor at 3 ng/ml, which stimulated the ECs to divide over a period of 3 days following administration [40].

This novel cellular biosensor may be useful for a number of purposes. One is the monitoring of different adherent cells in cell biology research projects where the viscoelastic properties of the cellular cytoskeleton and FACS-integrin attachment may be altered by a particular experimental manipulation. Another is in the screening of either small molecule drugs or macromolecular therapeutics' effects on adherent cells. For example, the biosensor can be used to screen small organic and inorganic molecules, oligonucleotides, peptides, polypeptides, carbohydrates, sugars, and other types and classes of candidate therapeutic agents or drugs. In particular, this biosensor would be of great utility for agents targeted to signal transduction elements, for example, the cytoskeleton, the membrane bound integrins, or the extracellular matrix of the cells used in the biosensor. The candidates that are shown to have a biological effect can be further screened, e.g., using cell-based assays or other in vitro assays. In addition, the positive candidates can be further screened in known animal models, e.g., for various cancers. Positive candidates can also be derivatized and formulated using known techniques to produce pharmaceutical drugs that can be administered to humans and animals by known routes of administration in known carriers and excipients.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

1. Sauerbray, G. Verwendung von Schwingquartzen zur Wagung dunner Schichten und zur microwagung, 2. Phys., 1959, 755, 206-222.

2. Bruckenstein, S.; Shay, M. In situ weighing study of the mechanism for the formation of the adsorbed oxygen monolayer at a gold electrode, J. Electroanal-Chem. 1985, 788, 131-136.

3. Bruckenstein, S.; Shay, M. Experimental Aspects of Use of the Quartz Crystal Microbalance in Solution, Electrochimica Acta, ,1985, 30, 1295-1300.

4. Kaufmnan, J. H.; Kanazawa. K. K.; Street, G. B., Gravimetric Electrochemical Voltage spectroscopy: In situ Mass Measurements during Electrochemical Doping of the Conducting Polymer Polypyrrole. Phys. Rev. Left., 1984, 53, 2461-2464.

5. Nomura, T.; Watanabe, M.; West, T-S.. Behavior of Pietoelectrlc Quartz Crystals in solutions with Application to the Determination of iodide., Anal. Chim. Acta. ,1985, 775. 107-116.

6. Kanazawa, K. K.: Gordon, G. The Oscillation Frequency of a Quartt Resonator in contact with a Liquid, Anal. Chim. Acta., 1985, 7 75, 99-105.

7. Muramatsu, H.; Kimura. K. Quartz Crystal Detector for Microrheological Study and Its Application to Phase Transition Phenomena of Langmuir-Blodgett Films, Anal. Chem., 1992, 64, 2502-2507.

8. Muramatsu, H.; Egawa, A.; Ataka, T. Reliability of correlation between mass change and resonant frequency change for a viscoelastic-film-coated quartz cry&, J., Electroanal. Chem., 1995, 388, 89-92.

9. Si, S-H.; Zhou, T.; Liu, D. Z.; Nie, L.; Yao, S. Using Piezoelectric Quartz Crystal 10 Study Hemorheological Phenomena: Blood Clotting and Urokinase Activated Fibrinoiysis, Anal. Letters, 1994, 27, 2027-2037.

10. Zhou, T.; Nie, L.; Shouzhou, Y., On equivalent circuits of piezoelectric quartz crystals in a liquid and liquid properties, J. Nectroanal. Chem., 1990, 293, 1-18. II, Fawcett, N.; Evans, J. A.; Chien, 1.; Flowers, N., Nucleic Acid Hybridization Detected by Piezoelectric Resonance, Anal. Letters, 1988, S1, 1099-11 14.

12. Su, H.; Yang, M.; Kallury, K. M. R.; Thompson, M., Interfacial Nucleic Acid Hybridization Studied by Random Primer 32P Labelling and Liquid Phase Acoustic Network Analysis, Anal. Chem., 1994, 66, 769-777.

13. Su. H.: Williams, P.; Thompson, M., Platinum Anticancer Drug Binding to DNA Detected by Thickness Shear Mode Acoustic Wave Sensor, Anal. Chem.. 1995, 67, 1010-1013.

14. fto, K.; Hashimoto, K.; lshimori, Y., Quantitative analysis for solid phase hybridization reaction. and binding reaction of DNA binder to hybrids using a quartz crystal microbalance, Anal. Chim. Acta., 1996, 327, 29-35.

15. Muratsugu, M.; Kurosawa, S.; Kamo, N.. Detection of Antistreptolysin 0 Antibody: Application of an Initial Rate Method of Latex Piezoelectric Immunoassay, Anal. Chem., 1992, 64, 2483-2487.

16. Ghouechian, H. O.; Kamo, N.; Hosokawa, T.; Akiiaya, T., Improvement of Latex Piezoelectric Immunoassay: Detection of Rheumatoid Factor, Talanta. 1994, 4 f, 401-406.

17. Thompson, M.; Arthur, G-L.; Dhaliwal, G-K., Liquid Phase Piezoelectric and Acoustic Transmission Studies of Interfacial Immunochemistry, Anal. Chem., 1986, 58, 1206. 1209.

18. Chang, H-C.; Yang, C. C.; Yeh, T. M., Detection of lipopolysaccharide binding peptides by the use of a lipopolysaccharide coated ‘piezoelectric crystal biosensor, Anal. Chim. Acta., 1997, 340, 49-54.

19. Fukuoka, S.; Karube, I.; influence of Cationlc Antibiotics on Phase Behavior of Rough Form Lipopolysaocharide, Applied Biochem. and Biotech., 1994, 43, 1-9.

20. Rickert, J.; Weiss, T.; Kraas, W.; Jung, G.; Gopel, W, A New Affinity Biosensor: Self-Assembled Thiols as Selective Monolayer Coatings of Quartz Crystal Microbalances, Biosensors &Bioelectronics, 1996, 7 7, 591-598.

21. Imai. S.; Mituno, H.; Suzuki, M.; Takeuchi, T.; Tamiya, E.; Mashige, F.; Ohkubo, A.; Karube, I., Total urinary protein sensor based on a plezoelectric quartz crystal. Anal . Chim. Acta., 1994, 292, 65-70.

22. Marx, K-A.: Zhou, T.; Sarma, R., Quartz Crystal Microbalance Measurement of SelfAssembled Micellar Tubules of the Amphiphilic Decyl Ester of D-Tyrosine and Their Enzymatic Polymerization, Biotechnology Progress, 1999, 7 5. 522-528.

23. Matsuda, T.; Kishida, A.; Ebato, H.; Okahata, Y., Novel Instrumentation Monitoring In situ Platelet Adhesivity with a Quartz Crystal Microbalance, ASA1 Journal, 1992, 38, 171-173.

24. Redepenning, J.; Schlesinger, T. K.; Mechalke, E. J.; Puleo, D. A.; Bizios, R., Osteoblast Attachment Monitored with a Quartz Crystal Microbalance, Anal. Chem., 1993, 65, 3378-3381,

25. Gryte, D-M.; Ward, M. D.; Hu, W. S., Real Tlme Measurement of Anchorage Dependent Cell Adhesion Using the Quartz Crystal Microbalance, Biofech. Progress, 1993, 9, 105-108.

26. Nivens, D. E.; Chambers, J. Q.; Anderson, T. R.; White, D. C., Long-Term On-Line Monitoring of Microbial Biofilms Using a Quartz Crystal Microbalance, Anal. Chem., 1993, 65, 65-69.

27. Muratsugu, M; Romanschin, A. D.; Thompson, M., Adhesion of human platelets to collagen detected by Cr51 labelling and acoustic wave sensor, Anal. Chimica Acta, 1997, 342, 23-29.

28. Wegener, J.; Janshoff. A.: Galla, H. J., Cell Adhesion monitoring using a quartz CWaf microbalance: comparative analysis of different mammalian cells lines, Eur. Biophys. J., 1998, 28, 26-37.

29. Fredriksson, C.; Kihlman, S., Rodahl, M.; Kasemo, B.. The Piezoelectric Quartz Crystal Mass and Dissipation Sensor: A Means of Studying Cell Adhesion, Langmuir, 1998, 14, 248-251.

30. Fredriksson, C.; Kihlman, S.; Rodahl, M.; Kasemo, B., In Vitro real-time characterization of cell attachment and spreading, J. Mater. Sci. Materials Medicine, . 1998, 9, 785-788.

31. Nicosia, R-F.; Villaschi, S., Autoregulation of angiogenesis by cells of the vessel wall, Int. Rev. Cytol., 1999, 785, 1-43.

32. Hungerford, J. E.: Little, C. D., Developmental Biology of the vascular smooth muscle ceil: building a multilayered vessel wall, J. Vast. Res., 1999, 36, 2-27.

33. Michel, C. C.: Curry, F. E., Microvascular Permeability, Physiol. Rev., 1999, 79, 703-761.

34. Engerman, R. L., Pfaffenbach, M. D., Davis, M. D., Cell Turnover of capillaries, Lab. Invest. 1967, 17, 738-741.

35. Weidner, N.; Semple. J. P., Welch, W. R.; Folkman, J., Tumor angiogenesis and metastasis correlation in invasive breast carcinoma, New Engl. J. Med., 1991, 324, 1-8.

36. Barakat, A-I., Responsiveness of vascular endothelium to shear stress: Potential role of ion channels and cellular cytoskelelon(Review), Int. J. Mol. Med., 1999, 4, 323.

37. Ingber, D-E., Tensegrity: the architectural basis of cellular mechanotransduction, Ann. Rev. Physiol., 1997, 53, 575-599.

38. DeBrabander, M. J.; Van de Viere, R. M. L.; Aerts, F. E. M.; Borgers, M.; Jannsen, P. A., The effects of Nocodazole, a New Synthetic Antitumoral Drug interfering with Microtubules, on Mammalian Cells Cultured in vitro, Cancer Research, 1976, 36, 905-916.

39. Zhou, T.; Braunhut, S-J.; Medeiros. D.: Marx, K. A., Cellular Adhesion and Spreading of Endothelial Cells Monitored in Real Time Using the Quartz Crystal Microbalance, In Tissue Engineeering Symposium: Materials Research Society, 1999, 550, 177-182.

40. Zhou, T.; Marx, K. A.; Warren, M.; Schulze, H.; Braunhut S-J,, The Quartz Crystal Microbalance as a Continuous Monitoring Tool for the Study of Endothelial Cell Surface Attachment and Growth, Biotechnology Progress, 2000, 76. 268-277.

41. Facci, J. S., Modification of Electrode Surfaces with Self-Organized Electroactive Microstructures, In Molecular Design o/Nedrode Surfaces, Techniques in Chemistry Series, Vol. XXII. ed. Royce W. Murray, John Wiley and Sons, Inc. 1992, 128-129..

42. Zhou. T.; Marx, K. A.; Warren, M.; Braunhut. S-J., Hydrophilic Modification of the Quark Crystal Microbalance Gold Surface Improves Endothelial Cell Adhesion and Spreading, Biotechnology Progress, submitted.

43. Atkins, P. W.. In Physical Chemistry, W. H. Freeman, San Francisco, Calif., 1978, 944.

44. Vasquez, R-J.; Howell, 5.; Yvon, A. M. C.; Wadsworth, P.; Cassimeris, L., Nanomolar Concentrations of Nocodazole Alter Microtubule Dynamic Instability in Vivo and In Vitro, Molecular Biology of the Cell, 1997, 8, 973-985

What is claimed:

1. A biosensor for detecting biological molecules in a quartz crystal microbalance (QCM), the biosensor comprising:
an endothelical cell (EC) matrix used as a biological signal transduction element; and
a piezoelectric mechanism for signal transduction to access attached EC cells as biological elements in the QCM biosensor.

2. The biosensor of claim 1, wherein the EC matrix adheres to a hydrophilically treated gold QCM surface under growth media containing serum.

3. The biosensor of claim 1, wherein the EC QCM biosensor detects EC cytoskeletal alterations.

4. A method of screening candidate drugs for their ability to affect cellular attachment, the method comprising applying the candidate drug to the surface of the EC matrix of the QCM biosensor of claim 1, and monitoring a change in the EC matrix.

5. The method of claim 4, wherein the candidate drug is a candidate anticancer therapeutic agent.
